(19) United States
(12) Patent Application Publication
(10) Pub. No.: US 2010/0239461 A1
(43) Pub. Date: Sep. 23, 2010

(54) Self-Running Sample Holder and System Having Self-Running Sample Holders

(75) Inventor: Teruaki Itoh, Kumamoto-shi (JP)

Correspondence Address: NIXON & VANDERHYE, PC, 901 NORTH GLEBE ROAD, 11TH FLOOR, ARLINGTON, VA, 22203, US

(73) Assignee: IDS Co., Ltd., Kumamoto (JP)

(21) Appl. No.: 12/793,918

(22) Filed: Jun. 4, 2010

Related U.S. Application Data
(62) Division of application No. 11/097,375, filed on Apr. 4, 2005.

(30) Foreign Application Priority Data
Apr. 7, 2004 (JP) 2004-113309

(51) Int. Cl. G01N 33/00

(52) U.S. Cl. 422/65

(57) ABSTRACT

A self-running sample holder includes a holder main body, a motor provided in the holder main body, a wheel, a transceiver and a battery. The holder main body has a holder portion which holds a sample with recorded information in an upright state. The wheel is provided in the holder main body and rotated in conjunction with the motor. The motor allows the self-running sample holder to self-run in an upright state. The transceiver is provided in the holder main body. The transceiver outputs a drive signal and drive stop signal to the motor in response to a signal from an external controller. The transceiver exchanges a signal with the controller. The battery is provided in the holder main body. The battery supplies the motor and transceiver with electricity. The battery is chargeable.

### CROSS-REFERENCE TO RELATED APPLICATIONS

This application is a divisional of U.S. patent application Ser. No. 11/097,375, filed Apr. 4, 2005, pending, which claims the benefit of Japanese Patent Application No. 2004-113309, filed Apr. 7, 2004, the entire contents of each of which are incorporated herein by reference.

### BACKGROUND OF THE INVENTION

1. Field of the Invention

The present invention relates to a sample holder which holds a sample, such as a test tube containing blood, and a sample holder conveyance system for conveying the sample holder to a predetermined position in order to apply predetermined processing to the sample.

2. Description of the Related Art

For example, there is available a holder conveyance apparatus that conveys a sample-vessel holder for holding a sample, such as a test tube containing blood, to a predetermined position in order to apply predetermined processing to the sample. Such a holder conveyance apparatus is disclosed in, for example, Jpn. Pat. Appln. KOKAI Publication No. 8-220105.

The holder conveyance apparatus disclosed in Jpn. Pat. Appln. KOKAI Publication No. 8-220105 includes, as a conveyance path for conveying a sample-vessel holder, a conveyor mechanism and a guide mechanism.

The conveyor mechanism is a belt-type conveyor that includes an endless belt and a motor to drive the endless belt. The conveyor mechanism conveys a sample-vessel holder for holding a sample. The guide mechanism includes side walls. The side walls are provided on both sides of the conveyor mechanism.

The holder conveyance apparatus further includes a bar-code reading unit for reading a bar-code printed on the sample at the portion where the conveyance path diverges. The bar-code reading unit selects the conveyance path to which the sample-vessel should be conveyed. Therefore, the holder conveyance apparatus includes a conveyance pause mechanism and an orientation control mechanism in order to read the bar-code.

The conveyance pause mechanism includes a piston section and an operating rod connected to the piston section. The conveyance pause mechanism inserts the operating rod in front of the moving direction of the sample-vessel holder to stop the movement of the sample-vessel holder. At this time, the sample-vessel holder is skidding on the belt.

The orientation control mechanism includes a piston section, a rod, and a press roller. The rod is contained in the piston section. The rod can be moved axially with respect to the piston section. The press roller is fitted to the distal end of the rod through a spring member.

The orientation control mechanism presses the press roller to the eccentric position of the top portion of the sample-vessel holder that has been stopped by the conveyance pause mechanism. As a result, a large frictional force is intensively generated at the eccentric position of the bottom portion of the sample-vessel holder and thereby the sample-vessel holder is rotated.

The rotation of the sample allows the bar-code printed on the sample to be read by the bar-code reading unit.

However, in the holder conveyance apparatus disclosed in Jpn. Pat. Appln. KOKAI Publication No. 8-220105, the sample-vessel holders are conveyed by the belt type conveyor mechanism. Thereby the structure of the holder conveyor mechanism is complicated.

In addition, in the holder conveyance apparatus disclosed in Jpn. Pat. Appln. KOKAI Publication No. 8-220105, a plurality of the orientation control mechanisms become required in the case where a plurality of diverging portions are provided on the conveyance path, complicating the structure of the holder conveyance apparatus.

### BRIEF SUMMARY OF THE INVENTION

An object of the present invention is to provide a self-running sample holder capable of simplifying the conveyance path and a conveyance system having the self-running sample holder capable of simplifying the structure thereof.

A self-running sample holder according to the present invention comprises a holder main body, a motor provided in the holder main body, a wheel, a transceiver and a battery. The holder main body has a holder portion which holds a sample with recorded information in an upright state. The wheel is provided in the holder main body and rotated in conjunction with the motor. The wheel allows the self-running sample holder to self-run in an upright state. The transceiver is provided in the holder main body. The transceiver outputs a drive signal and drive stop signal to the motor in response to a signal from an external controller. The transceiver exchanges a signal with the controller. The battery is provided in the holder main body. The battery supplies the motor and transceiver with electricity. The battery is chargeable.

The above configuration allows the self-running sample holder to self-run. Therefore, a mechanism such as a belt-type conveyor mechanism that conveys the sample does not need to be provided for the conveyance path for conveying the sample. As a result, the structure of the conveyance path for conveying the sample can be simplified.

In the preferred embodiment of the present invention, the transceiver is a wireless IC chip.

The adoption of the wireless IC chip as the transceiver makes the self-running sample holder independent from the controller. Therefore, the movement of the self-running sample holder is not impeded by the wiring.

A self-running sample holder conveyance system according to the present invention comprises a controller, a self-running sample holder, a conveyance path, a sample entrance portion, a sample exit portion, a plurality of processing section, a plurality of bypasses, a confirmation means, a reading unit, and a moving direction change mechanism.

The self-running sample holder comprises a holder main body, a motor provided in the holder main body, a wheel, a transceiver and a battery. The holder main body has a holder portion which holds a sample with recorded information in an upright state. The wheel is provided in the holder main body and rotated in conjunction with the motor. The wheel allows the self-running sample holder to self-run in an upright state. The transceiver is provided in the holder main body. The transceiver outputs a drive signal and drive stop signal to the motor in response to a signal from the controller. The transceiver exchanges a signal with the controller. The battery is provided in the holder main body. The battery supplies the motor and transceiver with electricity. The battery is chargeable.

The conveyance path guides the movement of the self-running sample holder. The sample entrance portion is provided in the conveyance path. The sample is held by the holder main body at the sample entrance portion. The sample exit portion is provided in the conveyance path. The sample is removed from the holder main body at the sample exit portion in the conveyance path. The plurality of processing sections are provided between the sample entrance portion and sample exit portion, in the conveyance path. The processing sections apply processing to the sample. The plurality of bypasses are provided in the conveyance path. The bypasses allow the self-running sample holder to bypass the processing sections. The confirmation means is provided in the conveyance path. The confirmation means transmits information indicating that the self-running sample holder has reached diverging portions between the conveyance path and the plurality of bypasses to the controller. The reading unit is provided in the conveyance path. The reading unit reads the recorded information before the self-running holder reaches one of the processing section, prior to the other processing section. The reading unit transmits the recorded information to the controller. The moving direction change mechanism is provided in the diverging portion. The moving direction change mechanism changes the moving direction of the self-running sample holder.

The controller causes the transceiver to store the recorded information received from the reading unit, confirms the recorded information stored in the self-running sample holder based on the information received from the confirmation means, and controls the moving direction change mechanism based on the confirmed information.

The above configuration enables the self-running sample holder to self-run. Therefore, it is not necessary to provide a mechanism for conveying the sample to the processing section in the conveyance path.

Further, the controller directly confirms the information of the sample stored in the transceiver at the diverging point between the conveyance path and bypasses. Therefore, it is not necessary to read the recorded information of the sample every time the self-running sample holder reaches the diverging portion. That is, it is not necessary to provide a mechanism that allows the self-running sample holder to rotate every time the self-running sample holder has reached the diverging portion, thereby simplifying the structure of the system.

In the preferred embodiment of the present invention, the self-running sample holder conveyance system comprises a charging section. The charging section is provided in the middle of the conveyance path. When the self-running sample holder reaches the charging section, the charging section is electrically connected to the battery to thereby charge the battery.

With the above configuration, the self-running sample holder can be charged simply by running on the conveyance path. Therefore, the self-running sample holder need not go out of the conveyance path for battery charge. That is, it is not necessary to provide a mechanism to guide the self-running sample holder to the outside of the conveyance path.

As a result, the structure of the system is simplified.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic configuration diagram of a self-running sample holder conveyance system according to an embodiment of the present invention;

FIG. 2 is a cross-sectional view of the self-running sample holder shown in FIG. 1;

FIG. 3 is a plan view showing a moving direction change mechanism shown in FIG. 1 in an enlarged manner;

FIG. 4 is a plan view showing a moving direction change mechanism shown in FIG. 1 in an enlarged manner; and

FIG. 5 is a cross-sectional view showing a charging section shown in FIG. 1 in an enlarged manner.

### DETAILED DESCRIPTION OF THE INVENTION

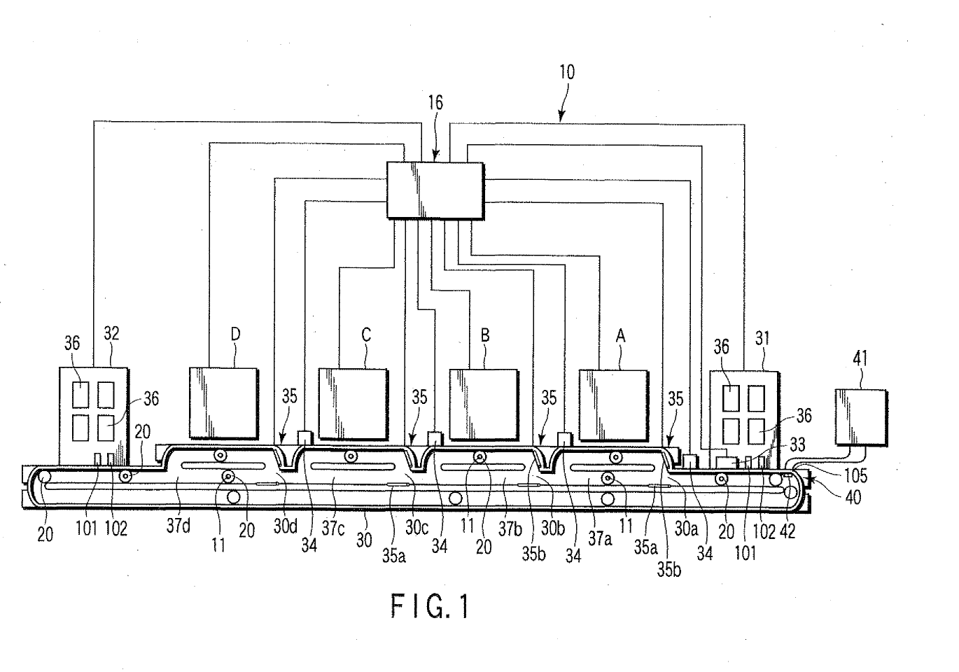

A self-running sample holder and a self-running sample holder conveyance system according to an embodiment of the present invention will be described with reference to FIGS. 1 to 5.

FIG. 1 is a schematic configuration diagram of a self-running sample holder conveyance system. As shown in FIG. 1, the self-running sample holder conveyance system 10 includes a controller 16, a self-running sample holder 20, a conveyance path 30, and a charging section 40.

The controller 16 controls the self-running sample holder conveyance system 10.

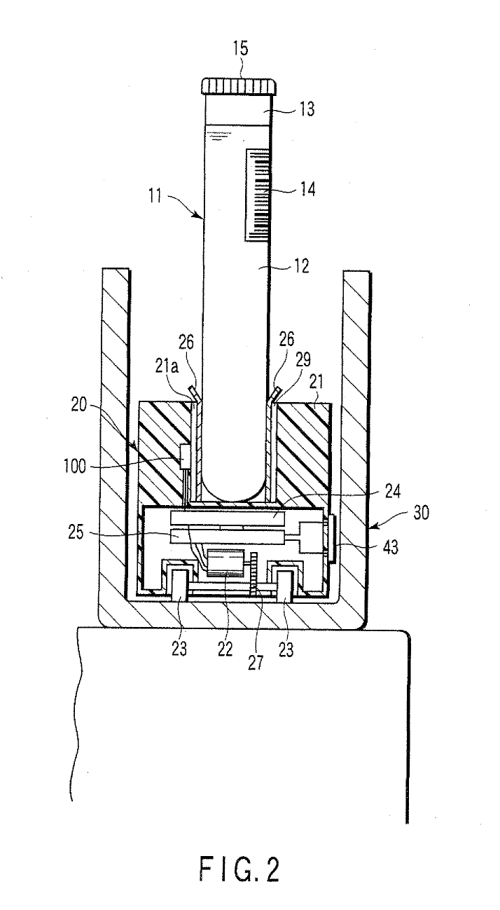

FIG. 2 is a cross-sectional view of the self-running sample holder shown in FIG. 1. As shown in FIG. 2, the self-running sample holder 20 includes a holder main body 21, a motor 22, a plurality of wheels 23, a wireless IC chip 24 serving as a transceiver, and a battery 25.

A housing hole 21a is formed in the holder main body 21. In the housing hole 21a, a sample 11 is housed. Therefore, the holder main body 21 has a substantially cylindrical shape. The holder main body 21 is made of, for example, synthetic resin.

The sample 11 is the concept includes blood 12 which is an example of an object to be examined and a test tube 13 which is an example of a containing vessel that contains blood 12.

A plug 15 is attached to the opening end of the test tube 13. A bar-code 14 is affixed to the outer circumferential surface of the test tube 13. The bar-code 14 is example of recorded information. Information such as reference number of the sample 11 and blood donor's name are recorded in the bar-code 14.

As shown in FIG. 2, a plurality of plate springs 26 are provided inside the housing hole 21a. The plate springs 26 are biased toward the inside of the housing hole 21a. The sample 11 is housed between the plate springs 26. The sample 11 is supported by the plate springs 26. As a result, the posture of the sample 11 is maintained in an substantially upright state in the housing hole 21a.

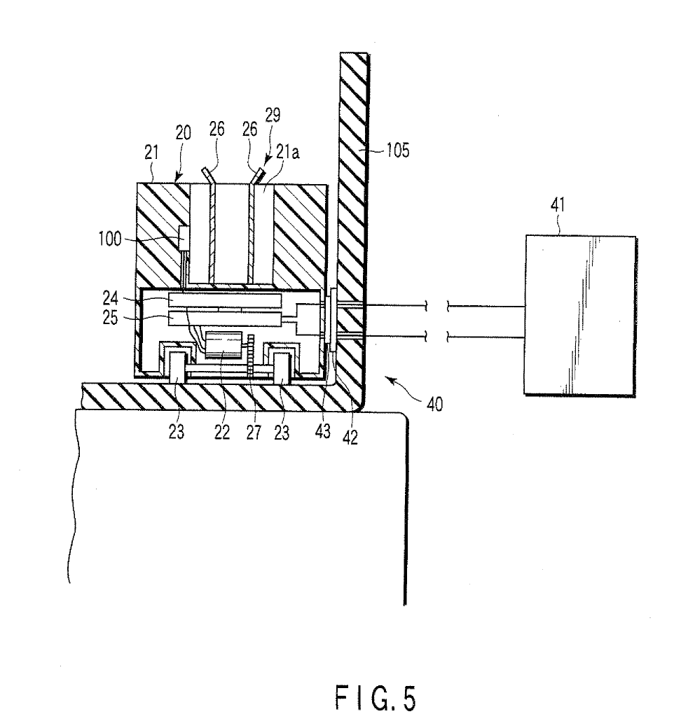

FIG. 5 shows a state where the sample 11 has been removed from the housing hole 21a. As shown in FIG. 5, the plate springs 26 can be moved toward the inside of the housing hole 21a. As a result, various-sized samples 11 can be held with their posture maintained in an upright state.

With the above configuration, the housing hole 21a and plate springs 26 constitute a holder portion 29 which holds the sample 11 in an upright state. The holder portion 29 is not limited to the configuration including the housing hole 21a and plate springs 26. It is sufficient for the holder portion 29 to be able to hold the sample 11 perpendicularly to the holder main body 21.

In a state where the sample 11 is held by the holder main body 21, the bar-code 14 is exposed outside from the holder main body 21.

The holder main body 21 further includes, for example, a hold-state confirmation sensor 100. The hold-state confirmation sensor detects that the sample 11 is held by the holder main body 21 and that the sample 11 is removed from the holder main body 21.

The motor 22 is provided inside the holder main body 21. The wheels 23 are provided in the lower portion of the holder main body 21. With the wheels 23, the self-running sample holder 20 can run with the posture of the holder main body 21 maintained in an upright state. The wheels 23 are connected to the motor 22 through a speed changer 27. Therefore, the self-running sample holder 20 can self-run.

The IC chip 24 is provided inside the holder main body 21. The IC chip 24 exchanges a signal with the controller 16 in wireless. The IC chip 24 outputs a drive signal and drive stop signal to the motor 22 in response to a signal from the controller 16. The IC chip 24 is electrically connected to the hold-state confirmation sensor 100. At the time when the sample 11 is held by the holder main body 21 or when the sample 11 is removed from the holder main body 21, the IC chip 24 transmits the above state to the controller 16 based on information from the hold-state confirmation sensor 100.

The battery 25 is provided inside the holder main body 21. The battery 25 is electrically connected to the motor 22 and IC chip 24. The battery 25 supplies the IC chip 24 and motor 22 with electricity. The battery 25 is chargeable.

As shown in FIG. 1, the self-running sample holder 20 can move on the conveyance path 30. The conveyance path 30 is formed in an annular shape. The conveyance path 30 includes a sample entrance portion 31, a sample exit portion 32, processing sections A to D, a reading unit 33, a confirmation sensor 34, and a moving direction change mechanism 35.

As shown in FIG. 1, the sample entrance portion 31 is provided, for example, at the right end portion on the conveyance path 30 in the figure. The sample entrance portion 31 is electrically connected to the controller 16. The sample entrance portion 31 is controlled by the controller 16. The sample entrance portion 31 contains a plurality of racks 36. The rack 36 contains, for example, fifty samples 11.

The sample entrance portion 31 includes, for example, a robot arm 101, a holder detection sensor 102, and the like. When the holder detection sensor 102 detects that the self-running sample holder 20 having no sample 11 has reached the sample entrance portion 31, the controller 16 allows the robot arm 101 to take the sample 11 out of the rack 36. The robot arm 101 then moves the taken sample 11 to the self-running sample holder 20.

In the sample entrance portion 31, the means for moving the sample 11 to the self-running sample holder 20 is not limited to the robot arm 101 and holder detection sensor 102. The sample 11 may be moved to the self-running holder 20 by another means.

As shown in FIG. 1, the sample exit portion 32 is provided, for example, at the left end portion on the conveyance path 30 in the figure. The sample exit portion 32 is electrically connected to the controller 16. Like the sample entrance portion 31, the sample exit portion 32 is controlled by the controller 16. More specifically, the sample exit portion 32 includes, for example, a robot arm 101, a holder detection sensor 102, and the like.

When the holder detection sensor 102 detects that the self-running sample holder 20 holding the sample 11 has reached the sample exit portion 32, the controller 16 allows the robot arm 101 to take the sample 11 out of the self-running sample holder 20. The robot arm 101 then moves the taken sample 11 to the rack 36. The configuration of the sample exit portion 32 is not limited to that including the robot arm 101, holder detection sensor 102, and the like.

The processing sections A to D are provided between the sample entrance portion 31 and sample exit portion 32 on the conveyance path 30. The processing sections A to D apply predetermined processing, such as examination, to the sample 11. Each of the processing sections A to D is electrically connected to the controller 16 and is controlled by the same.

Examples of the processing that the processing sections A to D perform include detecting a blood clot in the blood 12 of the sample 11, removing the plug 15 of the sample, and performing divided injection. The processing performed in the processing sections A to D is not limited to the above examples.

The number of the processing sections in the self-running sample holder conveyance system 10 is not limited. It is possible to appropriately incorporate the processing section according to need.

The reading unit 33 is provided inside the sample entrance portion 31. The reading unit 33 is electrically connected to the controller 16 and controlled by the same. The reading unit 33 reads the bar-code 14 on the sample 11 while the sample 11 is moved to the self-running sample holder 20. The reading unit 33 transmits the read information of the sample 11 to the controller 16.

The conveyance path 30 includes bypasses 37a to 37d which bypass the processing sections A to D. The bypasses 37a to 37d diverge from the conveyance path 30 at diverging portions 30a to 30d immediately before the processing sections A to D, respectively. After bypassing the processing sections A to D, each of the bypasses 37a to 37d converges to the conveyance path 30.

Note that the bypasses are provided to bypass the processing section. Therefore, when the number of the processing sections is increased, the number of the bypasses is increased correspondingly.

The confirmation sensors 34 are provided at immediately before the diverging portions 30a to 30d, respectively. Each of the confirmation sensors 34 detects that the self-running sample holder 20 has reached the corresponding diverging portion. The confirmation sensor 34 is an example of a confirmation means. Each of the confirmation sensors 34 is electrically connected to the controller 16. When detecting the self-running sample holder 20, the confirmation sensors 34 transmit detection information to the controller 16.

The moving direction change mechanisms 35 are provided in the diverging portions 30a to 30d, respectively. Each of the moving direction change mechanisms 35 is electrically connected to the controller 16. The moving direction change mechanisms 35 have a function of changing the moving direction of the self-running sample holder 20 under the control of the controller 16.

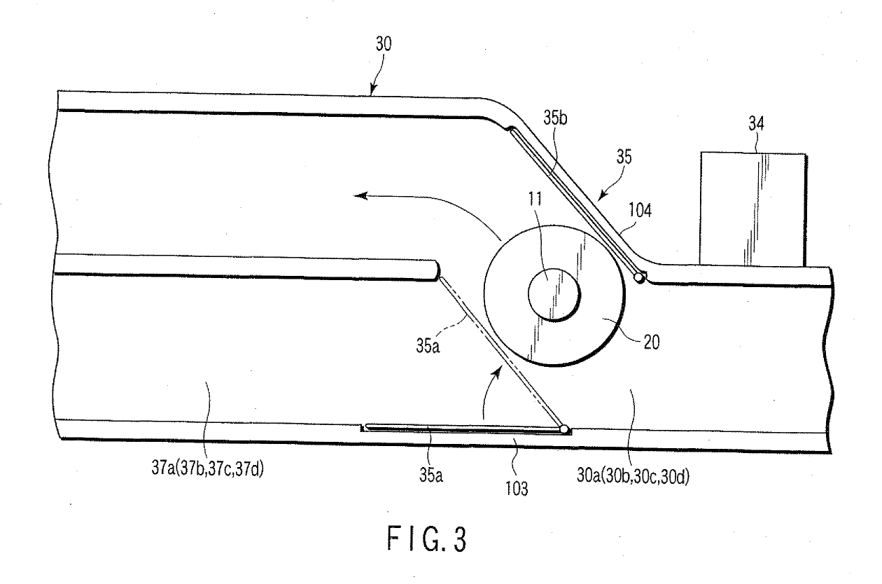

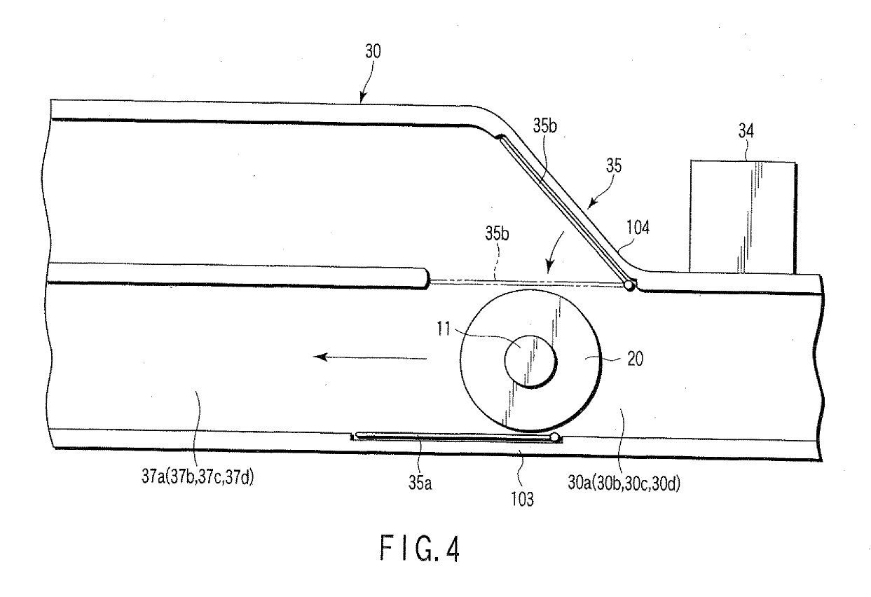

A description will be given of the moving direction change mechanism 35 with the moving direction change mechanism 35 provided in the diverging portion 30a taken as a representative. As shown in FIGS. 3 and 4, the moving direction change mechanism 35 includes a first gate 35a and a second gate 35b. The first gate 35a is provided in a vertical wall 103 on the bypass side. The first gate 35a can be opened and closed. As indicated by the chain double-dashed line in FIG. 3, when the first gate 35a is opened, the self-running sample holder 20 is prevented from entering the bypass 37a and is guided to the processing section A.

The second gate 35b is provided in a vertical wall 104 on the conveyance path side. The second gate 35b can be opened and closed. As indicated by the chain double-dashed line in FIG. 4, when the second gate 35b is opened, the self-running sample holder 20 is guided to the bypass 37a.

The moving direction change mechanisms 35 provided in the diverging portions 30b to 30d have the same function as above. The configuration of the moving direction change mechanism 35 is not limited to that including the first and second gates 35a and 35b as described above. It is sufficient for the moving direction change mechanism 35 to have the function of changing the moving direction of the self-running sample holder 20 in the diverging portions 30a to 30d.

As shown in FIG. 1, the charging section 40 is provided, for example, at the right end portion of the conveyance path 30 in the figure. FIG. 5 is a cross-sectional view of the charging section 40. As shown in FIG. 5, the charging section 40 includes a power source 41, a conveyance path side connector 42, and a holder side connection portion 43. The power source 41 is provided outside the conveyance path 30. The position of the power source 41 is not especially limited.

As shown in FIG. 1, the conveyance path side connector 42 is provided in the vertical wall 105 of the conveyance path 30. The conveyance path side connector 42 is electrically connected to the power source 41. As shown in FIG. 5, it is preferable that the vertical wall of the conveyance path 30 be made of an insulator. Only a portion of the vertical wall near the conveyance path side connector 42 may be made of an insulator.

As shown in FIGS. 2 and 5, the holder side connection portion 43 is provided on the outer circumferential surface of the holder main body 21. The holder side connection portion 43 is a conductor. The holder side connection portion 43 is electrically connected to the battery 25.

When the self-running sample holder 20 enters the charging section 40, the holder side connection portion 43 and conveyance path side connector 42 are electrically connected to each other. The electrical connection between the holder side connection portion 43 and conveyance path side connector 42 allows the power supply 41 to charge the battery 25.

A description will next be given of the operation of the self-running sample holder conveyance system 10. After being contained in the rack 36, the sample 11 is contained in the sample entrance portion 31. As shown in FIG. 1, when the self-running sample holder 20 enters the sample entrance portion 31, the holder detection sensor 102 detects the self-running sample holder 20. The holder detection sensor 102 then transmits the detection information to the controller 16.

Upon receiving the information from the holder detection sensor 102, the controller 16 allows the IC chip 24 to stop the drive of the motor 22. Then, the drive of the self-running sample holder 20 is stopped.

In the sample entrance portion 31, the sample 11 is moved to the self-running sample holder 20 under the control of the controller 16. At this time, the bar-code 14 affixed to the test tube 13 is read by the reading unit 33.

The reading unit 33 transmits the read information of the sample 11 to the controller 16. Upon receiving the information of the sample 11, the controller 16 transmits the information to the IC chip 24 of the self-running sample holder 20. The IC chip 24 stores the information after receiving the information of the sample 11. Assume that the sample 11 needs to be subjected to predetermined processing in the processing sections A and C.

When the hold-state confirmation sensor 100 confirms that the self-running sample holder 20 holds the sample 11, the IC chip 24 transmits the information indicating that the sample has been housed in the holder main body 21 to the controller 16. Upon receiving the above information, the controller 16 transmits a drive signal to drive the motor 22 to the IC chip 24. As a result, the motor 22 is driven and thereby the self-running sample holder 20 starts running.

When the confirmation sensor 34 confirms that the self-running sample holder 20 has reached immediately before the diverging portion 30a, the controller 16 confirms the information of the sample 11 stored in the IC chip 24 of the self-running sample holder 20 based on the information from the confirmation sensor 34.

The sample 11 needs to be subjected to predetermined processing in the processing section A. Therefore, the controller 16 opens the first gate 35a near the diverging portion 30a, as shown in FIG. 3. As a result, the self-running sample holder 20 is guided to the processing section A.

After being subjected to processing in the processing section A, the self-running sample holder 20 moves toward the diverging portion 30b. When the confirmation sensor 34 confirms that the self-running sample holder 20 has reached immediately before the diverging portion 30b, the controller 16 confirms the information of the sample 11 stored in the IC chip 24 of the self-running sample holder 20 based on the information from the confirmation sensor 34.

The sample 11 need not be subjected to processing in the processing section B. Therefore, the controller 16 opens the second gate 35b near the diverging portion 30b, as shown in FIG. 4. As a result, the self-running sample holder 20 is guided to the bypass 37b. The self-running sample holder 20 then bypasses the processing section B to move toward the diverging portion 30c.

When the confirmation sensor 34 confirms that the self-running sample holder 20 has reached the diverging portion 30c, the controller 16 confirms the information of the sample 11 stored in the IC chip 24 of the self-running sample holder 20 based on the information from the confirmation sensor 34.

The sample 11 needs to be subjected to predetermined processing in the processing section C. Therefore, the controller 16 opens the first gate 35a near the diverging portion 30c, as shown in FIG. 3. As a result, the self-running sample holder 20 is guided to the processing section C. The sample 11 of the self-running sample holder 20 is then subjected to processing in the processing section C.

After being subjected to processing in the processing section C, the self-running sample holder 20 moves toward the diverging portion 30d. When the confirmation sensor 34 confirms that the self-running sample holder 20 has reached immediately before the diverging portion 30d, the controller 16 confirms the information of the sample 11 stored in the IC chip 24 of the self-running sample holder 20 based on the information from the confirmation sensor 34.

The sample 11 need not be subjected to processing in the processing section D. Therefore, the controller 16 opens the second gate 35b near the diverging portion 30d, as shown in FIG. 3. As a result, the self-running sample holder 20 is guided to the bypass 37d. The self-running sample holder 20 then bypasses the processing section D to move toward the sample exit portion 32.

When the self-running sample holder 20 has reached the sample exit portion 32 as shown in FIG. 1, the holder detection sensor 102 detects the self-running sample holder 20. Upon receiving the detection information of the holder detection sensor 102 from the IC chip 24, the controller 16 allows the IC chip 24 to stop the drive of the motor 22.

As a result, the self-running sample holder 20 is stopped in the sample exit portion 32. Then the sample 11 is removed from the self-running sample holder 20. When the hold-state confirmation sensor 100 confirms the removal of the sample 11, the IC chip 24 transmits the information indicating the removal of the sample 11 to the controller 16.

Upon receiving the information indicating the removal of the sample 11, the controller 16 allows the IC chip 24 to drive the motor 22. As a result, the self-running sample holder 20 starts running toward the charging section 40.

As shown in FIG. 5, when the self-running sample holder 20 has reached the charging section 40, the holder side connection portion 43 and conveyance path side connector 42 are electrically connected to each other. As a result, the battery 25 is charged.

In the self-running sample holder conveyance system 10 having the configuration described above, the self-running sample holder 20 is self-running type. Therefore, a mechanism such as a belt-type conveyor mechanism that conveys the sample 11 need not be provided for the conveyance path 30, simplifying the structure of the conveyance path 30 of the self-running sample holder conveyance system 10.

Further, the IC chip 24 of the self-running sample holder 20 is a wireless type, eliminating the need to provide wiring for exchanging a signal with the controller 16. Therefore, the movement of the self-running sample holder 20 is not impeded by the wiring.

Further, the self-running sample holder 20 incorporates the IC chip 24 to store information related to the sample 11, thereby eliminating the need to read the bar-codes 14 of the samples 11 every time the samples 11 reach the diverging potions 30a to 30d.

That is, the self-running sample holder conveyance system 10 need not rotate the self-running sample holder 20 in order to read the bar-code 14. Therefore, it is not necessary to additionally provide a mechanism to rotate the self-running sample holder 20, simplifying the structure of the self-running sample holder conveyance system 10.

Further, in the self-running sample holder conveyance system 10, the charging section 40 is provided in the middle of the conveyance path 30. Therefore, the battery 25 is automatically charged while the self-running sample holder 20 is running.

In other words, in the case where the charging section 40 is provided outside the conveyance path 30, the self-running sample holder conveyance system 10 requires a mechanism to guide the self-running sample holder 20 to the charging section 40 provided outside.

In the present invention, however, it is not necessary to provide a mechanism to guide the self-running sample holder 20 to the outside of the conveyance path 30 for charging the battery, thereby simplifying the structure of the self-running sample holder conveyance system 10.

The processing sections A to D, sample entrance portion 31, sample exit portion 32, reading unit 33, confirmation sensor 34, and moving direction change mechanism 35 are electrically connected to the controller 16, respectively to exchange a signal between them in the above embodiment. However, the present invention is not limited to this, and the signal exchange may be performed by, for example, a wireless communication.

In order to prevent the self-running sample holders 20 from colliding with each other in the sample entrance portion 31, sample exit portion 32, and charging section 40, a means for detecting the self-running sample holder 20 may be provided in front of the sample entrance portion 31, sample exit portion 32, and charging section 40, respectively.

The information detected by the above means for detecting the self-running sample holder 20 is transmitted to the controller 16. The controller 16 may stop the self-running sample holder 20 based on the information. This prevents the self-running sample holders 20 from colliding with each other.

The sample 11 has been subjected to processing in the processing sections A and D in the above embodiment. However, the present invention is not limited to this. For example, in the case of another sample 11 to be subjected to processing in the processing sections B and D, the controller 16 controls the moving direction change mechanisms 35 such that the sample 11 is guided to the processing sections B and D.

As described above, the controller 16 controls the moving direction change mechanisms 35 such that the samples 11 are guided to the target processing sections.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A self-running sample holder conveyance system comprising:
a controller;
a self-running sample holder comprising: a holder main body having a holder portion which holds a sample with recorded information in an upright state;
a motor provided in the holder main body; a wheel to allow the holder main body to self-run in an upright state, the wheel being provided in the holder main body and rotated in conjunction with the motor; a transceiver provided in the holder main body, the transceiver outputting a drive signal and drive stop signal to the motor in response to a signal from the controller and exchanging a signal with the controller; and a battery to supply the motor and transceiver with electricity, the battery being provided in the holder main body and configured to be chargeable;
a conveyance path to guide the movement of the self-running sample holder;
a sample entrance portion at which the sample is held by the holder main body, the sample entrance portion being provided in the conveyance path;
a sample exit portion at which the sample is removed from the holder main body, the sample exit portion being provided in the conveyance path;
a plurality of processing sections to apply processing to the sample, the processing sections being provided between the sample entrance portion and sample exit portion in the conveyance path;
a plurality of bypasses to allow the self-running sample holder to bypass the processing sections, the bypasses being provided in the conveyance path;
confirmation means for transmitting information indicating that the self-running sample holder has reached diverging portions between the conveyance path and the plurality of bypasses to the controller, the confirmation means being provided in the conveyance path;
a reading unit which is provided in the conveyance path and which reads the recorded information before the self-running holder reaches one of the processing section, prior to the other processing sections, and transmits the recorded information to the controller;
a moving direction change mechanism to change the moving direction of the self-running sample holder, the moving direction change mechanism being provided in the diverging portion, wherein
the controller causes the transceiver to store the recorded information received from the reading unit, confirms the recorded information stored in the self-running sample holder based on the information received from the confirmation means, and controls the moving direction change mechanism based on the confirmed information.

2. The self-running sample holder conveyance system according to claim 1, further comprising a charging section provided in the middle of the conveyance path, the charging section being electrically connected to the battery to thereby charge the battery when the self-running sample holder reaches the charging section.
